(19) United States
(12) Patent Application Publication
(10) Pub. No.: US 2022/0133652 A1
(43) Pub. Date: May 5, 2022

(54) A FORMULATION FOR IMPROVING SEIZURE CONTROL

(71) Applicants: David MILLET, Emeryville, CA (US); ZOGENIX INTERNATIONAL LIMITED, Berkshire (GB)

(72) Inventors: David MILLET, Emeryville, CA (US); Bradley S. GALER, West Chester, PA (US)

(73) Assignees: ZOGENIX INTERNATIONAL LIMITED, Berkshire (GB); David MILLET, Emeryville, CA (US)

(21) Appl. No.: 17/430,033

(22) PCT Filed: Feb. 14, 2020
(86) PCT No.: PCT/US2020/018359

§ 371 Date: Aug. 11, 2021

Related U.S. Application Data
(60) Provisional application No. 62/810,228, filed on Feb. 25, 2019.

(51) Int. Cl. A61K 31/137; A61K 31/05; A61K 31/55; A61K 31/4168; A61K 31/135; A61K 31/197; A61K 31/19; A61K 31/277; A61K 31/5513; A61K 31/5517; A61K 31/53; A61K 9/00

(52) U.S. Cl. CPC A61K 31/137; A61K 31/05; A61K 31/55; A61K 31/4168; A61K 31/135; A61K 9/0019; A61K 31/19; A61K 31/277; A61K 31/5513; A61K 31/5517; A61K 31/53; A61K 31/197

(57) ABSTRACT

Described herein is a method of improving seizure control in a patient experiencing uncontrolled seizures persisting 10 minutes or more, comprising administering fenfluramine or a pharmaceutically acceptable salt, base, acid or amine thereof, at a dose of from 0.2 to 1.2 m/kg/day for a period of about 12 hours to about 7 days to a patient having been put into a therapeutic, medically-induced coma via a general anesthetic; and after about 12 hours to about 7 days, weaning the patient from the general anesthetic and assessing whether the seizure control has improved as compared to a pre-treatment time point. The patient experiencing seizures may have epilepsy or epileptic encephalopathy that has led to established status epilepticus (SE), refractory status epilepticus (RSE) or super-refractory status epilepticus (SRSE).

### FIELD OF THE INVENTION

Described herein is a method of treating patients with epilepsy or epileptic encephalopathy. In particular, the present disclosure is directed to a method of improving seizure control in a patient experiencing uncontrolled seizures persisting for 10 mins or more, depending on the type and/or absence of recovery of consciousness between seizures, comprising administering fenfluramine or a pharmaceutically acceptable salt, base, acid or amine thereof, at a dose of up to 60 mg/day and/or for a period of 12 hours up to 7 days to a patient in a medically-induced coma via a general anesthetic; and after 12 hours to 7 days, weaning the patient from the general anesthetic and assessing whether the seizure control has improved. The epilepsy or epileptic encephalopathy may be status epilepticus (SE), refractory status epilepticus (RSE) or super-refractory status epilepticus (SRSE), whether diagnosed or undiagnosed. Such patients are often put into a therapeutic, medically-induced coma via a general anesthetic; these patients are treated with fenfluramine, weaned from general anesthesia, and seizure control is assessed and may be better controlled as compared to a pre-treatment time point.

### BACKGROUND OF THE INVENTION

This invention relates to the treatment of epilepsy or epileptic encephalopathy and a method of improving seizure control in a patient experiencing uncontrolled seizures such as status epilepticus (SE), refractory status epilepticus (RSE) or super-refractory status epilepticus (SRSE), using an amphetamine derivative, specifically fenfluramine.

Fenfluramine, i.e. 3-trifluoromethyl-N-ethylamphetamine is an amphetamine derivative having the structure:

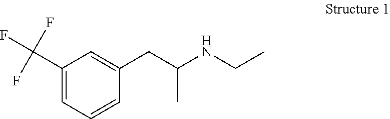 [SMILES: CCNC(C)Cc1cccc(C(F)(F)F)c1]

### (RS)-N-ethyl-1-[3-(trifluoromethyl)phenyl]propan-2-amine

Fenfluramine was first marketed in the US in 1973 and had been administered in combination with phentermine to prevent and treat obesity. However, in 1997, it was withdrawn from the US market as its use was associated with the onset of cardiac valvular fibrosis and pulmonary hypertension. Subsequently, the drug was withdrawn from sale globally and is no longer indicated for use in any therapeutic area anywhere in the world.

Despite the health concerns surrounding fenfluramine, attempts have been made to identify further therapeutic uses for that product. Aicardi and Gastaut (New England Journal of Medicine (1985), 313:1419 and Archives of Neurology (1988) 45:923-925) reported four cases of self-induced photosensitive seizures that responded to treatment with fenfluramine.

Clemens, in Epilepsy Research (1988) 2:340-343 reported a study on a boy suffering pattern sensitivity-induced seizures that were resistant to anticonvulsive treatment. Fenfluramine reportedly successfully terminated these self-induced seizures and the author concluded that this was because fenfluramine blocked the photosensitive triggering mechanism.

In Neuropaediatrics, (1996); 27(4):171-173, Boel and Casaer reported on a study on the effects of fenfluramine on children with refractory epilepsy. They concluded that when fenfluramine was administered at a dose of 0.5 to 1 mg/kg/day, this resulted in a reduction in the number of seizures experienced by the patients.

In a letter to Epilepsia, published in that journal (Epilepsia, 43(2):205-206, 2002), Boel and Casaer commented that fenfluramine appeared to be of therapeutic benefit in patients with intractable epilepsy.

Epilepsy is a condition of the brain marked by a susceptibility to recurrent seizures. There are numerous causes of epilepsy including, but not limited to birth trauma, perinatal infection, anoxia, infectious diseases, ingestion of toxins, tumors of the brain, inherited disorders, de novo gene mutations or degenerative disease, head injury or trauma, metabolic disorders, cerebrovascular accident and alcohol withdrawal.

Although the present invention has applicability with respect to a range of different types of epilepsies and epilepsy subtypes resulting in status epilepticus (SE), refractory status epilepticus (RSE) or super-refractory status epilepticus (SRSE), epilepsies of particular interest are Dravet syndrome, Doose syndrome, infantile spasms, Rett syndrome and Lennox-Gastaut syndrome.

A large number of subtypes of epilepsy have been characterized. For example, the most recent classification system adopted by the International League Against Epilepsy's (“ILAE”) Commission on Classification and Terminology provides the following list of epilepsy syndromes (See Berg et. al., “Revised terminology and concepts for organization of seizures,” Epilepsia, 51(4):676-685 (2010)):

I. Electroclinical syndromes arranged by age at onset:

A. Neonatal period (1. Benign familial neonatal epilepsy (BFNE), 2. Early myoclonic encephalopathy (EME), 3. Ohtahara syndrome)

B. Infancy (1. Epilepsy of infancy with migrating focal seizures, 2. West syndrome, 3. Myoclonic epilepsy in infancy (MEI), 4. Benign infantile epilepsy, 5. Benign familial infantile epilepsy, 6. Rett syndrome, 7. Myoclonic encephalopathy in nonprogressive disorders)

C. Childhood (1. Febrile seizures plus (FS+) (can start in infancy), 2. Panayiotopoulos syndrome, 3. Epilepsy with myoclonic atonic (previously astatic) seizures, 4. Benign epilepsy with centrotemporal spikes (BECTS), 5. Autosomal-dominant nocturnal frontal lobe epilepsy (ADNFLE), 6. Late onset childhood occipital epilepsy (Gastaut type), 7. Epilepsy with myoclonic absences, 8. Lennox-Gastaut syndrome, 9. Epileptic encephalopathy with continuous spike-and-wave during sleep (CSWS), 10. Landau-Kleffner syndrome (LKS), Childhood absence epilepsy (CAE))

D. Adolescence—Adult (1. Juvenile absence epilepsy (JAE), 2. Juvenile myoclonic epilepsy (JME), 3 Epilepsy with generalized tonic-clonic seizures alone, 4. Progressive myoclonus epilepsies (PME), 5. Autosomal dominant epilepsy with auditory features (ADEAF), 6. Other familial temporal lobe epilepsies

E. Less specific age relationship (1. Familial focal epilepsy with variable foci (childhood to adult), 2. Reflex epilepsies)

II. Distinctive constellations: A. Mesial temporal lobe epilepsy with hippocampal sclerosis (MTLE with HS), B. Rasmussen syndrome, C. Gelastic seizures with hypothalamic hamartoma, D. Hemiconvulsion-hemiplegia-epilepsy, E. Other epilepsies, distinguished by 1. presumed cause (presence or absence of a known structural or metabolic condition, then 2. primary mode of seizure onset (generalized vs. focal)

III. Epilepsies attributed to and organized by structural-metabolic causes:

A. Malformations of cortical development (hemimegalencephaly, heterotopias, etc.)

B. Neurocutaneous syndromes (tuberous sclerosis complex, Sturge-Weber, etc.)

C. Tumor

D. Infection

E. Trauma

IV. Angioma: A. Perinatal insults, B. Stroke, C. Other causes

V. Epilepsies of unknown cause

VI. Conditions with epileptic seizures traditionally not diagnosed as a form of epilepsy per se: A. Benign neonatal seizures (BNS); and B. Febrile seizures (FS)

See Berg et al., “Revised terminology and concepts for organization of seizures,” Epilepsia, 51(4):676-685 (2010)).

As can be seen from, for example, Part V of that list, there are still subtypes of epilepsy that have not yet been fully characterized and thus, the list is far from complete. For subtypes that are classified as encephalopathies, these conditions comprise a group of disorders in which seizure activity leads to progressive cognitive dysfunction.

Those skilled in the art will recognize that these subtypes of epilepsy are triggered by different stimuli, are controlled by different biological pathways and have different causes, whether genetic or environmental. In other words, the skilled artisan will recognize that teachings relating to one epileptic subtype are not necessarily applicable to other subtypes. This can include recognition that different epilepsy subtypes respond differently to different anticonvulsant drugs, where, for instance, one medicine may improve one condition while the same medicine may worsen another epilepsy condition.

Dravet syndrome is a rare and catastrophic form of intractable epilepsy that begins in infancy. Initially, in the first year of life the patient experiences prolonged seizures. In their second year, additional types of seizure begin to occur and this typically coincides with a developmental decline, possibly due to repeated seizures causing brain damage such as cerebral hypoxia. This then leads to poor development of cognition, language and motor skills.

Children with Dravet syndrome are likely to experience multiple seizures per day. Epileptic seizures are far more likely to result in death in sufferers of Dravet syndrome; approximately 10 to 15% of patients diagnosed with Dravet syndrome die in childhood, in some cases between two and four years of age. The mean age at death of patients is reported to be 8.7±9.8 years (SD), with 73% of deaths occurring before the age of 10 years, and 93% before the age of 20. Additionally, patients are at risk of numerous associated conditions including orthopedic developmental issues, impaired growth and chronic infections.

Frequent hospitalizations of children with Dravet syndrome are clearly distressing, not only to the patient but also to family and caregivers. The cost of care for Dravet syndrome patients is also high as the affected children require constant supervision and many require institutionalization as they reach teenage years.

At present, although a number of anticonvulsant therapies may be employed, singly or in combination, to reduce the instance of seizures in patients with Dravet syndrome, the results obtained with such therapies are typically poor and those therapies only effect partial cessation of seizures at best. Seizures associated with Dravet syndrome are typically resistant to conventional treatments. Further, many anticonvulsants such as clobazam and clonazepam have undesirable side effects, which are particularly acute and prominent in pediatric patients.

Of particular concern, children with Dravet syndrome are particularly susceptible to episodes of Status Epilepticus (SE), a convulsive seizure lasting longer than 5 minutes. Status epilepticus (SE) is a severe form of epilepsy. It is one of the most common neurologic emergencies, with an incidence of up to 61 per 100,000 per year and an estimated mortality of 20%. This severe and intractable condition is categorized as a medical emergency requiring immediate medical intervention, typically involving hospitalization for intravenous anticonvulsant medication and/or medically-induced coma. SE can be associated with severe cerebral hypoxia, possibly leading to damage to brain tissue; SE can be fatal. In some cases, SE progresses to refractory status epilepticus (RSE) and/or super-refractory status epilepticus (SRSE).

According to the American Society of Anesthesiology (ASA), a medically-induced coma occurs when a patient receives a controlled dose of general anesthesia, typically propofol, pentobarbital and/or thiopental, to cause a temporary coma or deep state of unconsciousness. Patients in medically-induced comas generally have brain injuries with swelling that have not responded to other treatments. The coma can protect the brain from swelling by reducing the metabolic rate of brain tissue and cerebral blood flow.

As many as 30%-40% of SE cases fail to respond to the standard treatment of antiepileptic medications and benzodiazepines. As a result, up to 44% of cases can progress to RSE, the second most common neurological emergency in the US, and require more extreme treatments be employed, such as a medically-induced coma, or “therapeutic coma.”

There is risk associated with refractory epilepsies that progress to SE, RSE or SRSE. Such emergencies require prompt and effective treatment, because the longer SE, RSE or SRSE goes on, the higher the chances of brain damage or the patient's body being unable to compensate for the trauma, leading to other complications like cardiac arrest or kidney or heart failure. The idea behind placing a patient into a therapeutic, medically-induced coma is to sedate the brain to a stage where there is no seizure activity, in effect, hitting the “reboot button” in the brain, letting the brain get some rest and have some time to reorganize itself. Furthermore, although experts agree that therapeutic coma is the proper treatment for RSE, it has yet to be elucidated how long patients should be kept in this state. Importantly, the longer a patient is maintained in a therapeutic coma, the higher the risk of complications.

Clinically, tonic-clonic convulsive SE is divided into four subsequent stages: early, established, refractory, and super-refractory. Thus, pharmacotherapy of SE can be based on a four-staged approach. However, data on alternative treatments in the later stages are limited; currently the data is sparse to support recommendations for most antiepileptic drugs for established, refractory, and SRSE. (Trinka, et al., (2015) Drugs. 75:1499-1521).

Early SE: (also known as premonitory status, impending status, and heraldic status). Phase I, early SE, occurs when the frequency and severity of seizures increases in a crescendo pattern. In patients without pre-existing epilepsy, the phase with a crescendo-like increase in seizure frequency and severity is missing, and SE starts abruptly. Ongoing convulsive epileptic activity for more than 5 min is now often called early SE. Initial treatment of early SE with intravenous lorazepam or intramuscular midazolam is able to control seizures in 63-73%; buccal midazolam may be an alternative whenever intravenous or intramuscular application of other benzodiazepines is not possible.

Established SE: Phase II, established SE, designates continuous seizure activity with convulsions, or intermittent seizures without regaining consciousness between seizures, or failure of initial treatment (usually benzodiazepines) of early SE. In established SE, seizures last more than 10 minutes and up to about 30 minutes. Despite a suboptimal safety profile, intravenous antiepileptic drugs (phenytoin/fosphenytoin, valproate, levetiracetam, phenobarbital) are most commonly used, but there is little to no evidence for choosing one over the other; alternatives to phenobarbital and phenytoin include valproate, levetiracetam, and lacosamide for treatment of established SE that persists despite first-line treatment with benzodiazepines.

Refractory SE (RSE): (also called advanced SE, subtle SE or stuporous SE). Phase III, known as RSE, refers to failed treatment of early and established SE (usually with antiepileptic drugs, AEDs). It is characterized by seizures increasing in duration (lasting from 30 min. to an hour), or the patient exhibits a decrease in motor activity (electromechanical dissociation) while the patient remains in a coma. Without being bound by theory, recent evidence from animal models (cholinergic agent-induced SE) indicates that seizure induced, maladaptive receptor trafficking may cause internalization (and temporary inactivation) of synaptic GABA_{A} receptors (GABA_{A}R) and an increase in synaptic NMDA receptors (NMDAR). These changes suggest a mechanism for the development of self-sustaining seizures independent of initial triggering cause or event (fewer GABA_{A}R and more NMDAR in synapses tilt the balance toward excitation and lessen inhibition thus establishing continuing seizures) and for pharmacoresistance (GABAergic drugs are less effective because they have fewer synaptic targets on which to act). (Niquet, et al., (2017) Epilepsia 58(4):e49-e53). Thus, failure to halt early or established SE progresses to self-sustaining seizures and more resistance to first line drugs used in treating RSE and SRSE.

Super-refractory status epilepticus (SRSE): (also known as “malignant SE”). In this fourth stage of SE, seizures continue despite maximal treatment with intravenous (IV) anesthetics for more than 24 h in an intensive care unit. These patients have ictal EEG discharges when anesthesia is lessened. Anesthetics (propofol, midazolam, thiopental/pentobarbital) are widely used in RSE and SRSE, with lower success rates and a high morbidity and mortality. Potential drugs and therapies being considered in treating SRSE are ketamine, magnesium, and immunomodulatory treatments, as well other cause-directed and non-medical treatments. (Trinka, et al., (2015) Drugs. 75:1499-1521).

Genetics and mechanisms of epilepsies: The most common mutation associated with Dravet syndrome is in the SCNA1 gene; the gene codes for the alpha-1 subunit of the sodium ion channel (Nav1.1), containing 2,009 amino acids, primarily expressed in inhibitory neurons. At least 70-80% of patients with Dravet syndrome have SCN1A mutations in the gene's exon which cause a loss of sodium channel function. Dravet has suggested as high as 85% have an SNC1A mutation (Dravet C. The core Dravet syndrome phenotype. Epilepsia 2011; 52 (Suppl. 2): 3-9). Some researchers predict that since only coding regions of the SCN1A gene are sequenced it is likely that many of the remaining patients harbor mutations in regulatory regions of the gene (outside of the coding sequences) that impair or prevent expression of this channel. Complete loss-of-function mutations in NaV1, encoded by SCNA1, cause Dravet Syndrome, which involves severe, intractable epilepsy and comorbidities of ataxia, sleep disturbance, and cognitive impairment. Mice with loss-of function mutations in NaV1.1 channels have severely impaired sodium currents and action potential firing in hippocampal GABAergic inhibitory neurons without detectable effect on the excitatory pyramidal neurons, which would cause hyperexcitability and contribute to seizures in Dravet Syndrome.

Impaired Nav1.1 channels, sodium currents and action potential firing are similarly impaired in the GABAergic Purkinje neurons in the cerebellum, which likely contributes to ataxia, and in the reticular nucleus of the thalamus and the suprachiasmatic nucleus of the hypothalamus, which likely contribute to circadian rhythm disturbances and sleep disorder. (Noebels et al., Jasper's Basic Mechanisms of the Epilepsies, 4th edition, Bethesda (Md.): National Center for Biotechnology Information (US); 2012).

Since mild loss-of-function mutations in NaV1.1 channels present a milder epilepsy phenotype called Familial Febrile Seizures, a unified loss-of-function hypothesis has been proposed for the spectrum of epilepsy syndromes caused by genetic changes in NaV1.1 channels: mild impairment predisposes to febrile seizures, intermediate impairment leads to GEFS+ epilepsy, and severe loss of function causes the intractable seizures and co-morbidities of Dravet Syndrome. (Catterall W A, et al., NaV1.1 channels and epilepsy. J. Physiol. 2010; 588: 1849-59).

Experts in the field were surprised that haploinsufficiency (in which only one functional copy of the gene, as opposed to the usual two) is not enough to maintain healthy neuronal network function of a NaV channel causes epilepsy, because reduced sodium current should lead to hypoexcitability rather than hyperexcitability. The mechanistic basis for hyperexcitability and co-morbidities in Dravet Syndrome was studied using an animal model generated by targeted deletion or mutation of the SCN1A gene in mouse. Homozygous null NaV1.1(−/−) mice developed ataxia and died on postnatal day (P) 15 Ogiwara, et al., J. Neurosci. 2007; 27:5903-5914, Yu, et al. Nat. Neurosci. 2006; 9:1142-1149. Heterozygous NaV1.1(+/−) mice exhibited spontaneous seizures and sporadic deaths beginning after P21, with a striking dependence on genetic background.

The loss of NaV1.1 did not change voltage-dependent activation or inactivation of sodium channels in hippocampal neurons, however, the sodium current density was substantially reduced in inhibitory interneurons of NaV1.1(+/−) and NaV1.1(−/−) mice, but not in their excitatory pyramidal neurons. This reduction in sodium current caused a loss of sustained high-frequency firing of action potentials in hippocampal and cortical interneurons, thereby impairing their in vivo inhibitory function that depends on generation of high-frequency bursts of action potentials.

Sodium channel blockers preferentially affect the sodium channel at a specific stage of its cycle of rest, activation and inactivation, often by delaying the recovery from the inactivated state, thereby producing a cumulative reduction of Na+.

Non-epileptic brains have a natural balance of excitation (that can evoke seizures) and inhibition (that can reduce seizures). In epilepsies that are caused by too much excitatory neurotransmission (many of the epilepsies except SCN1A mutation related epilepsies), sodium channel blockers are beneficial because they reduce the neurotransmitters that cause too much excitation.

Based on the mechanism in which sodium channel blockers work to prevent seizure activity, one would think that mutations in the SCN1A gene that cause the sodium channel to be ineffective (in essence, blocked) should prevent seizures and make a person with Dravet syndrome less prone to epilepsy. However, this loss of function is believed to lead to increased seizure activity, presumably because the result of this mutation is a decreased amount of inhibitory neurotransmitter that normally exists in the correct amount in the brain to balance excitatory neurotransmitters that make seizure more likely to occur. In this situation, the problem with the balance of excitation and inhibition in the brain is not too much excitation, it is too little inhibition. Giving sodium channel blocking drugs to Dravet syndrome patients further decreases the number of inhibitory neurotransmitters in the brain, tipping the balance toward more seizure activity.

In addition, it may be undesirable to treat the patient with any sodium channel drugs that are particularly undesirable when treating patients with Dravet syndrome. A certain class of drugs widely used in treating epilepsy, namely sodium channel blockers including carbamazepine, oxcarbazepine, lamotrigine, lacosamide, rufinamide, phenytoin, and fosphenytoin have been found to be contra-indicated in Dravet syndrome. These drugs may actually lead to a greater incidence of seizures and a worsened prognosis in almost all Dravet syndrome patients. Similarly, selective GABA reuptake inhibitors/GABA transaminase (“GABA T”) inhibitors including vigabatrin and tiagabine should be avoided in Dravet syndrome.

Sodium channel blocker drugs which may be contradicted in connection with the present invention may include the following: phenytoin, carbamazepine, lamotrigine, oxcarbazepine, rufinamide, lacosamide, eslicarbazepine acetate, and phosphenytoin.

Stiripentol is approved in Europe, Canada, Japan and Australia and was approved recently by the US FDA, for the treatment of Dravet syndrome. Possible mechanisms of action of stiripentol include direct effects mediated through the gamma-aminobutyric acid (GABA)A receptor and indirect effects involving inhibition of cytochrome P450 activity with resulting increase in blood levels of clobazam and its active metabolite. Stiripentol is labeled for use in conjunction with clobazam, and other antiepileptic drugs may be added such as valproate. However, concerns remain regarding the use of stiripentol due to its inhibitory effect on hepatic cytochrome P450 enzymes. Further, the interactions of stiripentol with a large number of drugs means that combination therapy (which is typically required for patients with Dravet syndrome) is problematic. Additionally, the effectiveness of stiripentol is limited, with few if any patients ever becoming seizure free.

Polypharmacy, the use of two or more anti-epileptic drugs, for the treatment of Dravet syndrome can result in a significant patient burden, as the side effects, or adverse events from the multiple medications can be additive, and result in limiting the effectiveness of the therapy due to intolerability; in other words the small benefit of a medication may not outweigh the risk or negative effects the drug is having on the patient. In many cases, available antiepileptic drugs do not offer adequate seizure control and respective neurosurgical procedures are not an option, and thus, new treatments for Dravet syndrome as well as SE, RSE and SRSE remain an important unmet need despite some level of efficacy in clinical trials for cannabidiol (Epidiolex®) and stiripentol (Diacomit®), which can be associated with cognitive or appetite safety concerns, respectively. Baraban, S, et al., Brain, 140 (3), p. 669-683 (March 2017).

Thus, there remains a need to provide a method for improving seizure control in a patient with status epilepticus (SE), refractory status epilepticus (RSE) or super-refractory status epilepticus (SRSE), who is experiencing persistent, uncontrolled seizures and who has been or will be placed in a medically-induced coma via a general anesthetic.

### SUMMARY OF THE INVENTION

The present disclosure is directed to the treatment of epilepsy or epileptic encephalopathy and a method of improving seizure control in a patient, using an amphetamine derivative, specifically fenfluramine. More particularly, the present disclosure provides a method of improving seizure control in a patient experiencing uncontrolled seizures persisting 10 mins or more depending on seizure type (e.g. convulsive) or recurring seizures without regaining consciousness, comprising administering fenfluramine or a pharmaceutically acceptable salt, base, acid or amine thereof, at a dose of 0.2 mg/kg/day to 1 mg/kg/day up to a maximum of 60 mg/day and/or for a period of about 12 hours to about 7 days to a patient in a medically-induced coma via a general anesthetic, and after about 12 hours to about 7 days, weaning the patient from the general anesthetic and assessing whether the seizure control has improved. The epilepsy or epileptic encephalopathy may be status epilepticus (SE), refractory status epilepticus (RSE) or super-refractory status epilepticus (SRSE), whether diagnosed or undiagnosed. The standard of care for patients experiencing SE, RSE or SRSE is to put the patient into a therapeutic, medically-induced coma via a general anesthetic for an indefinite period of time. With the present disclosure, SE, RSE and SRSE patients are treated with fenfluramine, which makes it possible to wean these patients from general anesthesia while maintaining seizure control as compared to a pre-treatment baseline and/or time point.

The present disclosure provides methods and formulations for use in treating a patient having SE, RSE or SRSE and in a medically-induced coma via a general anesthetic, comprising administering to a patient a therapeutically effective dose of fenfluramine or a pharmaceutically acceptable salt, base, acid or amine thereof which includes fenfluramine hydrochloride in a liquid formulation at a concentration of 1.25 mg/ml, 2.5 mg/ml or 5 mg/ml, providing the fenfluramine to the patient over a period of days, weeks or months on a dosing regimen of once a day, twice a day, three times a day or four times a day, wherein the dose is provided to the patient at an initial dose ranging from between 0.2 mg/kg/day and 5 mg/kg/day up to a maximum of 60 mg per day for up to about 5 days.

Preferably, the dosing is provided at twelve-hour intervals twice a day. With this treatment, it has been observed that a patient formerly refractive to treatment and thus maintained in a therapeutic, medically-induced coma can be weaned off the general anesthesia without appearance of break-through seizures that would indicate a need to resume administration of anesthesia.

In some embodiments of the method, fenfluramine is the sole therapeutic agent administered to the patient.

In some embodiments of the method, the fenfluramine is adjunctive therapy and is co-administered with a second, or a second and third, or a second, third and fourth, therapeutic agent. Any second, or any combination of second and third, or any combination of second, third and fourth therapeutic agents of interest may be utilized. In some cases, the second, or a second and third, or a second, third and fourth, therapeutic agent is selected from the group consisting of cannabidiol, carbamazepine, ethosuximide, fosphenytoin, lamotrigine, levetiracetam, phenobarbital, topiramate, stiripentol, valproic acid, valproate, verapamil, and benzodiazepines such as clobazam, clonazepam, diazepam, lorazepam, and midazolam and a pharmaceutically acceptable salt, base, acid or amine thereof.

In some embodiments, the method is adapted to a patient experiencing RSE or SRSE who overexpresses P-glycoprotein (P-gp) at the blood brain barrier (BBB) comprising administering a P-gp inhibitor as an additional agent. P-gp is an ATP-dependent cell surface transporter molecule that acts as an ATPase efflux pump for multiple agents, including many AEDs. P-gp actively pumps certain compounds, including many CNS drugs, out of cells. P-gp is encoded by the Adenosine triphosphate-binding cassette subfamily B member 1 (ABCB1) gene, also referred to as the multiple drug resistance 1 gene (MDR1). In some embodiments, RSE or SRSE causes increased P-gp levels in the BBB. In some embodiments the P-gp inhibitor is selected from verapamil, tariquidar, and elacridar.

In some embodiments, the fenfluramine treatment continues in amounts and over a period of time so as to reduce the need by the patient for maintaining the therapeutic, medically-induced coma by 25% or more, 50% or more, 75% or more, or completely eliminate the need for future inductions into a therapeutic coma state.

In another aspect of the invention, the treatment is continued in amounts and over a period of time so as to reduce the patient's hospitalization visits by 25% or more, 50% or more, 75% or more, or completely eliminate hospitalization visits due to refractory seizures.

Another aspect of the invention comprises administering a liquid fenfluramine formulation by the use of an oral syringe which is graduated for precise measurement of the liquid formulation. The formulation may include flavoring and coloring agents or may be completely devoid of any excipient materials beyond those necessary to dissolve the fenfluramine in the liquid which may be water.

In some cases, it can be desirable to test the patients for a genetic mutation prior to administration of some of the therapeutic agents, especially in cases where use of specific agent is contraindicated either because the agent is ineffective or because it would have undesired or serious side effects. Thus, it is in some cases desirable to test patients prior to treatment with fenfluramine. In the case of patients having Dravet syndrome, testing can be carried out for mutations in the SCN1A (such as partial or total deletion mutations, truncating mutations and/or missense mutations e.g. in the voltage or pore regions S4 to S6), SCN1 B (such as the region encoding the sodium channel β1 subunit), SCN2A, SCN3A, SCN9A, GABRG2 (such as the region encoding the γ2 subunit), GABRD (such as the region encoding the σ subunit) and I or PCDH19 genes have been linked to Dravet syndrome.

In some instances, the mutations occur in genes that are linked diseases and conditions characterized by various seizure types including, for example, generalized seizures, myoclonic seizures, absence seizures, and febrile seizures. Mutations may occur in one or more of the following genes: ALDH7A1, CACNA1A, CACNA1H, CACNB4, CASR, CHD2, CHRNA2, CHRNA4, CHRNB2, CLCN2, CNTN2, CSTB, DEPDC5, EFHC1, EPM2A, GABRA1, GABRB3, GABRD, GABRG2, GOSR2, GPR98, GRIN1, GRIN2A, GRIN2B, KCNMA1, KCNQ2, KCNQ3, KCTD7, MBD5, ME2, NHLRC1, PCDH19, PRICKLE1, PRICKLE2, PRRT2, SCARB2, SCN1A, SCN1B, SCN2A, SCN4A, SCN9A, SLC2A1, TBC1D24.

In some instances, the mutations occur in genes that are linked to age-related epileptic encephalopathies including, for example, early infantile epileptic encephalopathy. Mutations may occur in one or more of the following genes: ALDH7A1, ARHGEF9, ARX, CDKL5, CNTNAP2, FH, FOXG1, GABRG2, GRIN2A, GRIN2B, KCNT1, MAGI2, MAPK10, MECP2, NRXN1, PCDH19, PLCB1, PNKP, PNPO, PRRT2, RNASEH2A, RNASEH2B, RNASEH2C, SAMHD1, SCN1A, SCN1B, SCN2A, SCN8A, SCN9A, SLC25A22, SLC2A1, SLC9A6, SPTAN1, STXBP1, TCF4, TREX1, UBE3A, ZEB2.

In some instances, the mutations occur in genes that are linked to malformation disorders including, for example, neuronal migration disorders, severe microcephaly, pontocerebellar hypoplasia, Joubert syndrome and related disorders, holoprosencephaly, and disorders of the RAS/MAPK pathway. Mutations may occur in one or more of the following genes: AHI1, ARFGEF2, ARL13B, ARX, ASPM, ATR, BRAF, C12orf57, CASK, CBL, CC2D2A, CDK5RAP2, CDON, CENPJ, CEP152, CEP290, COL18A1, COL4A1, CPT2, DCX, EMX2, EOMES, FGF8, FGFR3, FKRP, FKTN, FLNA, GLI2, GLI3, GPR56, HRAS, INPP5E, KAT6B, KRAS, LAMA2, LARGE, MAP2K1, MAP2K2, MCPH1, MED17, NF1, NPHP1, NRAS, OFD1, PAFAH1B1, PAX6, PCNT, PEX7, PNKP, POMGNT1, POMT1, POMT2, PQBP1, PTCH1, PTPN11, RAB3GAP1, RAF1, RARS2, RELN, RPGRIP1L, SHH, SHOC2, SIX3, SLC25A19, SNAP29, SOS1, SPRED1, SRD5A3, SRPX2, STIL, TGIF1, TMEM216, TMEM67, TSEN2, TSEN34, TSEN54, TUBA1A, TUBA8, TUBB2B, VDAC1, WDR62, VRK1, ZIC2.

In some instances, the mutations occur in genes that are linked to epilepsy in X-linked intellectual disability. Mutations may occur in one or more of the following genes: ARHGEF9, ARX, ATP6AP2, ATP7A, ATRX, CASK, CDKL5, CUL4B, DCX, FGD1, GPC3, GRIA3, HSD17B10, IQSEC2, KDM5C, MAGT1, MECP2, OFD1, OPHN1, PAK3, PCDH19, PHF6, PLP1, PQBP1, RAB39B, SLC16A2, SLC9A6, SMC1A, SMS, SRPX2, SYN1, SYP.

In some instances, the mutations occur in genes that are linked to storage diseases and conditions characterized by organelle dysfunction including, for example, neuronal ceroid lipofuscinosis, lysosomal storage disorders, congenital disorders of glycosylation, disorders of peroxisome biogenesis, and leukodystrophies. Mutations may occur in one or more of the following genes: AGA, ALG1, ALG12, ALG2, ALG3, ALG6, ALG8, ALG9, ALG11, ALG13, ARSA, ARSB, ASPA, B4GALT1, CLN3, CLN5, CLN6, CLN8, COG1, COG4, COG5, COG6, COG7, COG8, CTSA, CTSD, DDOST, DOLK, DPAGT1, DPM1, DPM3, EIF2B1, EIF2B2, EIF2B3, EIF2B4, EIF2B5, FUCA1, GALC, GALNS, GFAP, GLB1, GNE, GNPTAB, GNPTG, GNS, GUSB, HEXA, HEXB, HGSNAT, HYAL1, IDS, IDUA, MCOLN1, MFSD8, MGAT2, MLC1, MOGS, MPDU1, MPI, NAGLU, NEU1, NOTCH3, NPC1, NPC2, PEX1, PEX12, PEX14, PEX2, PEX26, PEX3, PEX5, PEX6, PEX7, PEX10, PEX13, PEX16, PEX19, PGM1, PLP1, PMM2, PPT1, PSAP, RFT1, RNASEH2A, RNASEH2B, RNASEH2C, SAMHD1, SDHA, SGSH, SLC17A5, SLC35A1, SLC35A2, SLC35C1, SMPD1, SUMF1, TMEM165, TPP1, TREX1.

In some instances, the mutations occur in genes that are linked to syndromic disorders with epilepsy including, for example, juvenile myoclonic epilepsy, childhood absence epilepsy, benign rolandic epilepsy, Lennox-Gastaut syndrome, Dravet syndrome, Ohtahara syndrome, West syndrome, etc. Mutations may occur in one or more of the following genes: ATP2A2, ATP6VOA2, BCKDK, CACNA1A, CACNB4, CCDC88C, DYRK1A, HERC2, KCNA1, KCNJ10, KIAA1279, KMT2D, LBR, LGI1, MAPK10, MECP2, MEF2C, NDE1, NIPBL, PANK2, PIGV, PLA2G6, RAIL RBFOX1, SCN8A, SERPINI1, SETBP1, SLC1A3, SLC4A10, SMC3, SYNGAP1, TBX1, TSC1, TSC2, TUSC3, UBE3A, VPS13A, VPS13B.

In some instances, the mutations occur in genes that are linked to the occurrence of migraines. Mutations may occur in one or more of the following genes: ATP1A2, CACNA1A, NOTCH3, POLG, SCN1A, SLC2A1.

In some instances, the mutations occur in genes that are linked to Hyperekplexia. Mutations may occur in the following genes: ARHGEF9, GLRA1, GLRB, GPHN, SLC6A5.

In some instances, the mutations occur in genes that are linked to inborn errors of metabolism including, for example, disorders of carbohydrate metabolism, amino acid metabolism disorders, urea cycle disorders, disorders of organic acid metabolism, disorders of fatty acid oxidation and mitochondrial metabolism, disorders of porphyrin metabolism, disorders of purine or pyridine metabolism, disorders of steroid metabolism, disorders of mitochondrial function, disorders of peroxisomal function, and lysosomal storage disorders. Mutations may occur in one or more of the following genes: ABAT, ABCC8, ACOX1, ACY1, ADCK3, ADSL, ALDH4A1, ALDH5A1, ALDH7A1, AMT, ARG1, ATIC, ATP5A1, ATP7A, ATPAF2, BCS1L, BTD, C120RF65, CABC1, COQ2, COQ9, COX10, COX15, DDC, DHCR7, DLD, DPYD, ETFA, ETFB, ETFDH, FOLR1, GAMT, GATM, GCDH, GCSH, GLDC, GLUD1, GLUL, HPD, HSD17B10, HSD17B4, KCNJ11, L2HGDH, LRPPRC, MGME1, MMACHC, MOCS1, MOCS2, MTHFR, MTR, MTRR, NDUFA1, NDUFA2, NDUFAF6, NDUFS1, NDUFS3, NDUFS4, NDUFS7, NDUFS8, NDUFV1, PC, PDHA1, PDHX, PDSS1, PDSS2, PGK1, PHGDH, POLG, PRODH, PSAT1, QDPR, RARS2, SCO2, SDHA, SLC19A3, SLC25A15, SLC46A1, SLC6A8, SUCLA2, SUOX, SURF1, TACOLTMEM70, VDAC1.

Other genetic tests can be carried out and/or can be required as a condition of treatment.

In some embodiments, the one or more targets are selected from the group consisting of the sigma-1 receptor, the 5-HT_{1A} receptor, the 5-HT_{1D} receptor, the 5-HT_{2A} receptor, the 5-HT_{2C} receptor, and the SERT transporter.

In some embodiments, disclosed herein is a method of improving seizure control in a patient experiencing uncontrolled seizures persisting 30 mins or more and placed in a medically-induced coma via a general anesthetic, said method comprising administering fenfluramine or a pharmaceutically acceptable salt, base, acid or amine thereof, at a dose of about 0.2 mg/kg/day up to a dose 1.0 mg/kg/day provided the maximum dose does not exceed 60 mg and/or for a period of about 12 hours to about 7 days and after between 12 hours and 7 days, then weaning the patient from the general anesthetic and assessing whether the seizure control has improved. In some embodiments, the method further comprises determining the patient had previously failed treatment with stiripentol and/or cannabidiol based on lack of efficacy or based on tolerability. In some embodiments, the fenfluramine administration is repeated over a period of a week or more or more, administering the fenfluramine twice per day in a liquid formulation in an initial dose ranging from between 0.2 mg/kg/day to 5 mg/kg/day up to a maximum of 60 mg per day for up to about 5 days until the patient can be weaned from general anesthesia and the patient's seizures are eliminated over a period of 10 days or more.

### DETAILED DESCRIPTION OF THE INVENTION

Before the present methods of treatment are described, it is to be understood that this invention is not limited to particular method described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

Each of the patents, patent applications and articles cited herein is incorporated by reference. It must be noted that as used herein and in the appended claims, the singular forms “a”, “an”, and “the” include plural referents unless the context clearly dictates otherwise; the use of the article “a” or “an” is intended to encompass one or more. Thus, for example, reference to “a step of administering” includes a plurality of such steps and reference to “the symptom” includes reference to one or more symptoms and equivalents thereof known to those skilled in the art, and so forth.

The foregoing description and the examples are intended as illustrative and are not to be taken as limiting. Still other variations within the spirit and scope of this invention are possible and will readily present themselves to those skilled in the art.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The phrase “medically-induced coma” or “therapeutic coma” refers to a temporary coma or deep state of unconsciousness brought on by administration of a controlled dose of general anesthesia to a patient, typically propofol, pentobarbital and/or thiopental, in attempt to allow brain inflammation and/or swelling to subside and seizures to become better controlled (i.e., reduced from baseline frequency and/or severity).

The term “reduction from baseline” is used throughout in order to refer to a reduction relative to the same or similar patient prior to administration of fenfluramine. During the baseline period, the patient is treated with other therapeutic agents, except for fenfluramine. Treatment with the same other therapeutic agents is substantially maintained during the treatment with fenfluramine. The comparison is made relative to the observations, measurements or tests made during the baseline period.

The term “fenfluramine” refers to both the free-base depicted in Structure 1 above as well as its pharmaceutically acceptable acid addition salts. Pharmaceutically acceptable acid addition salts are those formed from acids which form non-toxic acid anions such as, for example, the hydrochloride, hydrobromide, sulphate, phosphate or acid phosphate, acetate, maleate, fumarate, lactate, tartrate, citrate and gluconate salts. The term “ZX008” refers to fenfluramine hydrochloride formulated as an oral solution.

### Specific Aspects of the Invention

After many years of extensive research, it has unexpectedly been found that fenfluramine can administered as described here to reduce or eliminate seizures in patients with epilepsy or epileptic encephalopathies, including Dravet syndrome. This is confirmed by the results presented herein. Additional information is in the article by Ceulemans et al., Epilepsia (2012) 53(7):1131-1139, the contents of which are incorporated herein.

For the avoidance of doubt, the term “prevention” of seizures means the total or partial prevention (inhibition) of seizures. Ideally, the methods of the present invention result in a total prevention of seizures; indeed, this ideal has been achieved in a number of patients treated by the inventors. However, the invention also encompasses methods in which the instances of seizures are decreased by at least 50%, at least 60%, at least 70%, at least 80% or at least 90%.

It is known that patients with Dravet syndrome commonly experience photosensitive or induced seizures. From teachings in the prior art, e.g. Aicardi and Gastaut (1988) and Boel and Casaer (1996)—both discussed above—it might have been expected that fenfluramine would reduce photosensitive or induced seizures. Importantly, however, it has surprisingly been found that all types of seizures exhibited by patients with Dravet syndrome, that is seizures in addition to and other than those that are photosensitive or induced, convulsive and non-convulsive can be suppressed by treatment in accordance with a method of the present invention. Convulsive seizures involve the entire body and are involuntary; they include a sudden onset of very evident, intense rapid muscle contraction (tonic phase) and followed by jerking of extremities (clonic phase) of body muscles, and also may include shaking, loss of consciousness, difficulty breathing, loss of bowel/bladder control and/or confusion. usually lasting a few minutes. Atonic seizures are a type of seizure that causes sudden loss of muscle strength, also called akinetic seizures, drop attacks or drop seizures in which the sudden lack of muscle strength, or tone, can cause the person to fall to the ground and are typically classified as a type of motor seizure. Atonic seizures occur commonly in patients having Lennox Gastaut syndrome. The affected person usually remains conscious and may not fall, but may exhibit head drop, drooping eyelids, or they may drop anything they were holding.

Seizures that lack clonic or tonic activity or other major motor activity are classified as non-convulsive and they may range from being readily apparent to being nearly undetectable by an observer. Non-motor focal seizures with or without impaired awareness can involve sensory, cognitive, emotional or autonomic abnormalities depending on the area of the brain experiencing seizure activity.

Atypical absence seizures are so named because they are of longer duration and have a slower onset and offset than absence seizures (i.e., the more usual sort of impaired awareness seizure) and involve different symptoms. Atypical absence seizures may begin with staring into space, usually with a blank look accompanied usually by a change in muscle tone and movement. Repetitive blinking may occur which appears as rapid fluttering of the eyelids. Automatisms such as smacking of the lips or chewing movements, rubbing fingers together or making other hand motions may also occur which are not under the voluntary control of the patient. An atypical absence seizure can last up to 20 seconds or more.

Thus, in context of the present invention, the term “seizure” is used to not only encompass photosensitive or induced seizures, but some or all of the other types of seizures experienced by patients with epilepsy

Moreover, fenfluramine's therapeutic effects appear to be independent of any significant placebo effects. In general, the effects of the placebo arm in epilepsy clinical trials are generally quite positive, making an efficacious therapy difficult to validate. While seizure-freedom rates on placebo are quite low (0-2.8%), rates on 50%-responder rates on placebo are quite a bit larger (4-27%) (Burneo et al., 2002; Cramer et al., 1999; Guekht et al., 2010; Rheims et al., 2008; Zaccara et al., 2015), and may be higher yet due to a statistically significant publication bias in epilepsy public trials (Beyenburg et al., 2010). Although the placebo phenomenon may be partially attributable to normal disease progression (Goldenholz et al., Ann. Neurol. 2015 SEP; 78(3): 329-336. Published online 2015 Jul. 29, doi 10.1002/ana.24470), and its magnitude influenced by a number of factors, it is verifiable, and likely due to positive or negative expectations of patients and of investigators. See generally Goldenholz et al., Response to Placebo in Clinical Epilepsy Trials—Old Ideas and New Insights Epilepsy Res. 2016 May; 122: 15-25, Published online 2016 Feb. 10. doi: 10.1016/j.eplepsyres.2016.02.002.

Unexpectedly, the results obtained in double-blinded fenfluramine clinical trials effectively match those from open label studies, which leads to the surprising conclusion that fenfluramine's efficacy is free of any placebo effect, unlike the majority of more conventional anti-epileptics. This is an unexpected and surprising result providing improvements in the reliability and robustness of fenfluramine's efficacy as an antiseizure medication in treatment of epilepsies and epileptic encephalopathies.

Thus, according to a further aspect of the present invention, there is provided a method of improving seizure control in a patient experiencing SE, RSE or SRSE and in a therapeutic, medically-induced coma via a general anesthetic, by administering to that patient a therapeutically effective dose of fenfluramine, leading to the ability to wean the patient from the general anesthetic. In various embodiments of this aspect, no break-through seizures are observed during the first hour, the second hour, the third hour, the sixth hour, and/or the twelfth hour after the patient begins to emerge from the induced coma and regain consciousness. In other embodiments, the instances of seizures upon weaning from the general anesthetic are decreased by at least 50%, at least 60%, at least 70%, at least 80% or at least 90%.

Thus, according to a further aspect of the present invention, there is provided a method of treating a patient experiencing SE, RSE or SRSE that exhibits a mutation in one, some or all of the above genes by administering to that patient an effective dose of fenfluramine. In certain embodiments of this aspect of the invention, the patient has been diagnosed with Dravet syndrome.

Fenfluramine has been known to inhibit serotonin reuptake and to trigger the release of serotonin in the brain due to disruption of its vesicular storage. Data from more recent studies provide evidence that fenfluramine is a positive allosteric modulator of the sigma-1 receptor. The results provided here indicate a high degree of efficacy in the treatment of refractive epilepsies requiring induction of a therapeutic coma via general anesthesia, by administering fenfluramine to dramatically reduce and in some cases completely eliminate seizures from patients being treated and thereby allowing the patient to be weaned from general anesthesia.

Thus, according to an aspect of the present disclosure, a method is provided for improving seizure control in a patient experiencing uncontrolled seizures persisting 10 minutes or repeated convulsive seizures without regaining consciousness, comprising administering fenfluramine or a pharmaceutically acceptable salt, base, acid or amine thereof, at a dose of 0.2 mg/kg/day up to 1.0 mg/kg/day provided that the daily dose does not exceed 60 mg/day and/or for a period of 12 hours to about 7 days to a patient in a medically-induced coma via a general anesthetic; and after about 12 hours to about 7 days, weaning the patient from the general anesthetic and assessing whether the seizure control has improved.

In some embodiments of the method, the assessing of seizure control is measured by ECG, video-electroencephalographic telemetry and continuous observation by trained personnel, or any combination thereof.

In some embodiments, the weaning is started about 12 hours to about 7 days after starting administration of fenfluramine.

In some embodiments, the assessment indicates that seizure control has not improved adequately, and the method further comprises reestablishing the medically-induced coma with a general anesthetic, and increasing the dose of fenfluramine.

In some embodiments, the general anesthetic is chosen from propofol, a barbiturate, midazolam and ketamine, or any combination thereof. In some embodiments, the general anesthetic is a barbiturate selected from pentobarbital and thiopental.

In some embodiments, seizure control has improved sufficiently to continue weaning the patient from general anesthesia while continuing to administer fenfluramine. After the initial early doses ranging from between 0.2 mg/kg/day and 5 mg/kg/day up to a maximum of 60 mg per day for up to about 5 days, the continuing administration of fenfluramine can be titrated down to between 0.2 mg/kg/day and 0.8 mg/kg/day for maintenance of improved seizure control conditions.

In some embodiments, the patient has been diagnosed with refractory status epilepticus (RSE) or super-refractory status epilepticus (SRSE).

In some embodiments, the patient has been diagnosed with a disease or condition selected from an epilepsy or epileptic encephalopathy (e.g., Dravet syndrome, Doose syndrome, infantile spasms, Lennox-Gastaut syndrome, Rett syndrome); attentional disorders (e.g., attention deficit disorder (ADD) or attention deficit/hyperactivity disorder (ADHD)); developmental disorders, such as autism spectrum disorders (ASDs), including autism, Asperger syndrome, pervasive developmental disorder (PDD) and pervasive developmental disorder not otherwise specified (PDD-NOS); oppositional defiant disorder (ODD); learning disabilities (e.g. dyslexia, dyscalculia); Tourette syndrome; traumatic brain injury; lead exposure; anxiety and/or depression; and low birth weight; or any combination thereof. In some embodiments, the patient has been diagnosed with Dravet syndrome. In some embodiments, the patient has been diagnosed with Lennox-Gastaut syndrome.

In some embodiments, the fenfluramine is formulated with a pharmaceutically acceptable carrier, and an effective dose is selected from the group consisting of less than about 10.0 mg/kg/day, less than about 5.0 mg/kg/day, less than 1.0 mg/kg/day, about 0.8 mg/kg/day, and about 0.5 mg/kg/day.

In some embodiments, fenfluramine is used as an adjunctive therapy in the patient.

In some embodiments, at least one co-therapeutic agent is administered, wherein said agent is selected from the group consisting of Brivaracetam, bromides (e.g., Potassium Bromide, Sodium Bromide), Cannabidiol, Carbamazepine, Clonidine, Ergenyl Chrono, Ethosuximide, Felbamate, Fosphenytoin, Ketamine, Lacosamide, Lamotrigine, Levetiracetam, Levocarnitine, Mesuximide, Nitrazepam, Oxcarbazepine, Perampanel, Phenobarbital, Pregabalin, Progabide, Pyridoxine, Rufinamide, Sultiame, Tizanidine, Topiramate, Stiripentol, Valproate semisodium, Valproate sodium, Valproic acid, Verapamil, Zonisamide, and benzodiazepines such as Clobazam, Clonazepam, Diazepam, Ethyl Loflazepate, Lorazepam and Midazolam, and a pharmaceutically acceptable salt, base, acid or amine thereof.

In some embodiments, the patient is maintained on a ketogenic diet or vagus nerve stimulation.

In some embodiments, fenfluramine is in a dosage form for a route of administration selected from the group consisting of oral, injectable, transdermal, inhaled, nasal, rectal, vaginal and parenteral. In some embodiments, the dosage form is in the form of an intravenous solution. In some embodiments, the dosage form is a solution for oral administration via a gastric feeding tube.

In some embodiments an intubated or comatose patient is administered a parenteral dosage form of fenfluramine or a pharmaceutically acceptable salt thereof. Exemplary forms of parenteral administration include intravenous, intraarterial, subcutaneous, intramuscular, intravascular, intraperitoneal or infusion. In some embodiments, the fenfluramine is formulated for subcutaneous administration. Fenfluramine and its pharmaceutically acceptable salts can be incorporated for administration by injection into aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline can also be used for injection. Exemplary excipients include sterile water for injection, ethanol, glycerol, propylene glycol, liquid polyethylene glycol, cyclodextrin derivatives, and vegetable oils.

Sterile injectable solutions can be prepared by incorporating fenfluramine or a pharmaceutically acceptable salt thereof in the required amount in the appropriate solvent with one or more excipients, such as, for example, buffering agents to provide a suitable pH, followed by filtered sterilization. Dispersions can be prepared by incorporating sterilized fenfluramine or a pharmaceutically acceptable salt thereof into a sterile vehicle. An injectable formulation can be sterilized, for example, by filtration through a bacterial-retaining filter, or by incorporating sterilizing agents in the form of sterile solid compositions which can be dissolved or dispersed in sterile water or other sterile injectable medium prior to use. Injectable compositions can contain from about 0.1 to about 5% w/w of fenfluramine or a pharmaceutically acceptable salt thereof.

Although it is not essential, in an embodiment, benzyl alcohol may be incorporated in the composition up to a concentration of 7%. The benzyl alcohol has the desirable properties of exerting an anti-bacterial action and also of providing a localized anesthetic effect upon parenteral administration of the drug.

In some embodiments, the improved seizure control is observed as a decrease in frequency, length or severity, and/or an increase in the length of time between seizures, as compared to the frequency, length, severity, or length of time between seizures in a patient not treated with fenfluramine and weaned from the general anesthesia.

In some embodiments of the method, after the patient is treated with fenfluramine, the time between seizures increases, and is selected from at least 0.5 hours, at least 1 hour, at least 1.5 hours, at least 2 hours, at least 3 hours, at least 4 hours, at least 5 hours, at least 6 hours, at least 12 hours, at least 24 hours, at least 2 days, at least 3 days, at least 4 days, at least 5 days, at least 6 days, at least 1 week, at least 2 weeks, at least 3 weeks, at least 4 weeks, at least 2 months, at least 6 months, and at least 1 year.

In some embodiments, the patient is 18 years of age or younger. In some embodiments, the patient is over 18 years of age.

In some aspects, a kit is provided, wherein the kit comprises a fenfluramine formulation, a package, and a package insert comprising instructions for use in improving seizure control in a patient experiencing uncontrolled seizures.

In some aspects, a kit is provided, wherein the kit comprises a container comprising a plurality of doses of a formulation comprising a pharmaceutically acceptable carrier and an active ingredient comprising fenfluramine, and instructions for treating a patient experiencing uncontrolled seizures with the formulation and assessing the patient's ability be weaned off general anesthesia after treatment with the formulation.

The dose of fenfluramine administered in the methods of the present invention can be formulated in any pharmaceutically acceptable dosage form including, but not limited to oral dosage forms such as tablets including orally disintegrating tablets, capsules, lozenges, oral solutions or syrups, oral emulsions, oral gels, oral films, buccal liquids, powder e.g. for suspension, and the like; injectable dosage forms; transdermal dosage forms such as transdermal patches, ointments, creams; inhaled dosage forms; and/or nasally, rectally, vaginally administered dosage forms. Such dosage forms can be formulated for once a day administration, or for multiple daily administrations (e.g. 2, 3 or 4 times a day administration).

Dosage may be based on patient's body weight, and ranges specified herein are inclusive of endpoints, unless otherwise specified.

The dosage form of fenfluramine employed in the methods of the present invention can be prepared by combining fenfluramine with one or more pharmaceutically acceptable diluents, carriers, adjuvants, and the like in a manner known to those skilled in the art of pharmaceutical formulation.

In some embodiments, fenfluramine can be employed as a monotherapy. Alternatively, fenfluramine can be co-administered simultaneously, sequentially or separately with one or more co-therapeutic agents, such as anticonvulsants. Such agents can be selected from the group consisting of cannabidiol, carbamazepine, ethosuximide, fosphenytoin, lamotrigine, levetiracetam, phenobarbital, progabide, topiramate, stiripentol, valproic acid, valproate, verapamil, and benzodiazepines such as clobazam, clonazepam, diazepam, ethyl loflazepate, lorazepam, midazolam. Use of a pharmaceutically acceptable salt, base, acid or amine of a co-therapeutic agent is also contemplated. However, carbamazepine, oxcarbazepine, lamotrigine, phenytoin and vigabatrin are typically contraindicated in Dravet syndrome, as they tend to make seizures worse, rather than better.

Fenfluramine can be employed to treat a patient who has previously been treated with an anticonvulsant, e.g., as described herein, such as stiripentol or cannabidiol. In some instances, the patient has been diagnosed with Dravet syndrome, Doose syndrome, infantile spasms, Lennox-Gastaut syndrome or Rett syndrome that is refractory to treatment with one or more anticonvulsant agents as described herein. In certain instances, the anticonvulsant agent is a modulator of neuronal GABA(A) receptors, such as stiripentol. By refractory to anticonvulsant agent (e.g., stiripentol or cannabidiol) is meant that the frequency of convulsive seizures (CSF) is not significantly reduced in the patient in response to therapy (e.g., monotherapy) with the anticonvulsant agent. In some cases, a significant reduction in CSF is a 10% or greater reduction in mean monthly convulsive seizures, such as 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, or 99% or greater reduction. In certain instances, the subject method is a method of improving seizure control in a patient diagnosed with Dravet syndrome refractory to stiripentol. In various embodiments, the instances of seizures (e.g., mean monthly convulsive seizures) are decreased by at least 50%, at least 55%, at least 60%, at least 65%, at least 70%, at least 75%, at least 80%, at least 85%, at least 90%, or at least 95%.

The invention includes a use as described throughout, wherein the fenfluramine is the only active ingredient administered to the patient.

In some aspects, provided herein is a method of improving seizure control in a patient experiencing uncontrolled seizures persisting 10 mins or more or repeated convulsive seizures without regaining consciousness, comprising administering fenfluramine or a pharmaceutically acceptable salt, base, acid or amine thereof, at a dose of 0.2 mg/kg/day up to 1.0 mg/kg/day provided that the daily dose does not exceed 60 mg/day and/or for a period of 12 hours to about 7 days to a patient in a medically-induced coma via a general anesthetic, and after about 12 hours to about 7 days, weaning the patient from the general anesthetic and assessing whether the seizure control has improved. The epilepsy or epileptic encephalopathy may be status epilepticus (SE), refractory status epilepticus (RSE) or super-refractory status epilepticus (SRSE), whether diagnosed or undiagnosed. The standard of care for patients experiencing SE, RSE or SRSE is to put the patient into a therapeutic, medically-induced coma via a general anesthetic for an indefinite period of time. Typically, physicians will attempt to wean refractory patients from anesthesia after 24 hours, and if the seizures return, the patients are re-administered general anesthesia for 5- to 7-day cycles followed by additional weaning attempts, which will typically lead to resolution if the underlying disorder resolves. With the present disclosure, SE, RSE and SRSE patients are treated with fenfluramine, which makes it possible to wean these patients from general anesthesia while maintaining seizure control as compared to a pre-treatment time point. When fenfluramine is administered as a trial agent in controlling super refractory status epilepticus attempts at weaning may be made at a shorter interval than 5 to 7 days after initiation, such as starting to wean after between about 12 hours and about 5 days or between about 1 and about 4 days. In some embodiments, the administering is repeated over a period of a day or days, or weeks until the patient can be weaned from the general anesthetic while exhibiting improved seizure control as compared to baseline as measured by a decrease in convulsive seizure frequency or seizure severity or seizure length, and/or an increase in time between seizures. In some embodiments, the method further comprises repeating the administering until the patient is seizure free for a period of ≥1 day, or for a period of ≥9 days, or for a period of ≥14 days, or for a period of ≥21 days, or for a period of ≥14 weeks, or for a period of ≥6 months, or for a period of ≥1 year. In some embodiments, the method further comprises repeating the administering until the patient is permanently seizure free. In some embodiments of the method, convulsive seizures are completely eliminated for 10 days or more, 20 days or more, 30 days or more, 50 days or more, 100 days or more. In some embodiments of the method, the repeating administration continues over a period of 4 weeks or more until a significant reduction from baseline in convulsive seizure frequency is observed after the patient has been weaned from general anesthesia.

In some embodiments, the administering is over a period of months, and the co-therapeutic agent is clobazam. In some embodiments, the co-therapeutic agent is a combination of stiripentol, valproate and clobazam. Administration may be daily, once a day, twice a day, three times a day or four times a day. In some embodiments, the dose is provided to the patient at a level of an initial dose ranging from between 0.2 mg/kg/day and 5 mg/kg/day up to a maximum of 60 mg per day for up to about 5 days. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments, the treatment improves two or more symptoms selected from the group consisting of convulsive seizures, ataxias, gait abnormalities, sleep disturbances and cognitive impairment. In some aspects, the present disclosure provides a method of improving seizure control in a patient in a medically-induced coma via a general anesthetic, comprising administering to the patient a therapeutically effective dose of fenfluramine or a pharmaceutically acceptable salt, base or acid thereof in an amount ranging from between 0.2 mg/kg/day and 5 mg/kg/day up to a maximum of 60 mg per day for up to about 5 days; administering a co-therapeutic agent; and repeating the administering of the co-therapeutic agent and fenfluramine over a period of weeks until the patient can be weaned from the general anesthetic and maintain improved levels of seizure control, exhibited, for example, as a reduction from baseline in convulsive seizure frequency of 40% or more, 50% or more, or 60% or more. Pharmaceutical compositions and formulations for use in practicing the subject methods are also provided. In some embodiments, the method comprises repeated administrations of fenfluramine (or salt, acid, base or amine thereof) over a period of days until the patient can be weaned from the general anesthetic and maintain improved levels of seizure control, exhibited as an increase from baseline in an average time between convulsive seizures of eight hours or more.

In some embodiments, the method comprises a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; administering a co-therapeutic agent; and the co-therapeutic agent and fenfluramine are in a liquid formulation for use in repeated daily administrations over a period of weeks until the patient can be weaned from the general anesthetic and maintain improved levels of seizure control, exhibited as an increase from baseline in average time between convulsive seizures of one week or more.

In some embodiments, as described throughout, the method comprises fenfluramine as the only active ingredient administered to the patient.

In some embodiments, the post-fenfluramine treatment is observed to increase the average time between seizures in a human patient, comprising administering to the patient a therapeutically effective dose of fenfluramine or a pharmaceutically acceptable salt, base, acid or amine thereof, and repeating the administering over a period of days until the patient can be weaned from the general anesthetic and maintain improved levels of seizure control, exhibited as an increase from baseline in average time between convulsive seizures of six hours or more, or an average time of eight hours or more, or an average time of one day or more, or an average time of two days or more, or an average time of one week or more, or an average time of one month or more. In some embodiments, a patient in a medically-induced coma via a general anesthetic is treated by administering to the patient a therapeutically effective dose of fenfluramine or a pharmaceutically acceptable salt, base, acid or amine thereof in an amount an initial dose ranging from between 0.2 mg/kg/day and 5 mg/kg/day up to a maximum of 60 mg per day for up to about 5 days; administering a co-therapeutic agent; and repeating the administering of the co-therapeutic agent and fenfluramine over a period of weeks until the patient can be weaned from the general anesthetic and maintain improved levels of seizure control, exhibited, for example, as an increase from baseline in average time between convulsive seizures of 6 to 23 hours or more, 1 to 6 days or more, 1 to 3 weeks or more, 1 to 11 months or more, one year or more, or seizures are completely eliminated for 10 days or more, 20 days or more, 30 days or more, 50 days or more, 100 days or more. In some embodiments, the administering is repeated over a period of days until the patient exhibits an increase from baseline in average time between convulsive seizures of 4 hours or more, 5 hours or more, 6 hours or more, 7 hours or more, 8 hours or more, 9 hours or more, 12 hours or more, 15 hours or more, 18 hours or more, or 24 hours or more. In some embodiments, repeating the administering occurs over a period of a day or days, or over a period of weeks, or over a period of months or over a period of years. In some embodiments in which the repeat administration is daily, the administration is once a day, twice a day, three times a day or four times a day. In some embodiments, the dose is provided to the patient at a level ranging from between 0.2 mg/kg/day and 5 mg/kg/day up to a maximum of 60 mg per day for up to about 5 days. In some embodiments, the administering is over a period of months, and the co-therapeutic agent is clobazam. In some embodiments, the co-therapeutic agent is a combination of stiripentol, valproate and clobazam. In some embodiments, the method further comprises repeating the administering until the patient is seizure free for a period of ≥1 day, or for a period of ≥9 days, or for a period of ≥14 days, or for a period of ≥21 days, or for a period of ≥14 weeks, or for a period of ≥6 months, or for a period of ≥1 year. In some embodiments, the method further comprises repeating the administering until the patient is permanently seizure free. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml. In some embodiments, the fenfluramine hydrochloride in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments of the method, the repeating administration continues over a period of 4 weeks or more until an increase from baseline in average time between convulsive seizures of 6 to 23 hours or more, 1 to 6 days or more, 1 to 3 weeks or more, 1 to 11 months or more, one year or more, or seizures are completely eliminated for 10 days or more, 20 days or more, 30 days or more, 50 days or more, 100 days or more is observed. In some embodiments, the therapeutically effective dose of fenfluramine or a pharmaceutically acceptable salt, base, acid or amine thereof is twice per day in a liquid formulation in an amount an initial dose ranging from between 0.2 mg/kg/day and 1.5 mg/kg/day up to a maximum of 60 mg per day for up to about 5 days.

In some embodiments, the method comprises a therapeutically effective dose of fenfluramine or a pharmaceutically acceptable salt, base, acid or amine thereof, wherein the use is for repeated administrations over a period of days until the patient can be weaned from the general anesthetic and maintain improved levels of seizure control, exhibited, for example, as a reduction from baseline in a seizure type experienced by the patient.

In some embodiments, the method comprises a therapeutically effective dose of fenfluramine or a pharmaceutically acceptable salt, base, acid or amine thereof, in an amount an initial dose ranging from between 0.2 mg/kg/day and 5 mg/kg/day up to a maximum of 60 mg per day for up to about 5 days; a co-therapeutic agent; and the co-therapeutic agent and fenfluramine are in a liquid formulation for use in repeated daily administrations over a period of weeks until the patient can be weaned from the general anesthetic and maintain improved levels of seizure control, exhibited, for example, as a reduction from baseline of two types of seizures.

In some aspects, provided herein is a method of reducing a particular type of seizure in a human patient experiencing uncontrolled seizures, by administering to the patient a therapeutically effective dose of fenfluramine or a pharmaceutically acceptable salt, base, acid or amine thereof, and repeating the administering over a period of a day or days, or over a period of weeks, months or years until the patient can be weaned from the general anesthetic and maintain improved levels of seizure control, exhibited, for example, as a significant reduction (e.g., 40%, 50% 60%, 70%, 80%, 90%, 95% or even greater) from baseline in seizures of a particular type. In some embodiments, a method is provided for treating a patient diagnosed with Dravet syndrome, by administering to the patient a therapeutically effective dose of fenfluramine or a pharmaceutically acceptable salt, base, acid or amine acid thereof; and repeating the administering over a period of days until the patient exhibits a reduction from baseline in a seizure type experienced by the patient. The reduction may be of one, two, three or multiple specific types of seizures. In some embodiments, two seizure types are reduced. In some embodiments, three seizure types are reduced. In some embodiments of the method, the seizure type reduced is selected from the group consisting of non-convulsive seizures, generalized seizures, myoclonic seizures, absence/atypical absence seizures, and febrile seizures, or any combination thereof. In some embodiments, particularly in Dravet syndrome, multiple seizure types are typically present including convulsive seizures consisting of generalized clonic seizures (GCS), generalized tonic-clonic seizures (prior terminology was grand mal), or alternating unilateral clonic seizures; myoclonic seizures; atypical absences and obtundation (dulled or impaired awareness) status; focal seizures, with or without secondary generalization; or, more rarely, tonic seizures. In some embodiments, the seizure type reduced is selected from the group consisting of photosensitive seizures and self-induced seizures. In some embodiments, the seizure type reduced is selected from atonic, or focal seizures without clear observable motor signs. In some embodiments, the method further comprises recording the seizure types experienced daily by the patient or caregiver in an electronic diary. In some embodiments, repeating the administering occurs over a period of a day or days, or over a period of weeks, or over a period of months or over a period of years. In some embodiments in which the repeat administration is daily, the administration is once a day, twice a day, three times a day or four times a day. In some embodiments, the dose is provided to the patient at a level ranging from between 0.2 mg/kg/day and 5 mg/kg/day up to a maximum of 60 mg per day for up to about 5 days. In some embodiments, the method further comprises repeating the administering of the fenfluramine in an amount of 0.2 mg/kg/day or more up to 60 mg/day until the patient no longer experiences at least one type of seizure experienced by the patient prior to administering the fenfluramine. In some embodiments, the method further comprises repeating the administering of the fenfluramine in an amount of 0.2 mg/kg/day or more up to 60 mg/day until the patient improves two or more symptoms selected from the group consisting of convulsive seizures, ataxias, gait abnormalities, sleep disturbances and cognitive impairment. In some embodiments, the patient exhibits a reduction from baseline in a particular seizure type of 50% or more, 60% or more, 70% or more, 80% or more, 90% or more, 95% or more. In some embodiments, the particular seizure type is completely eliminated for 10 days or more, 20 days or more, 30 days or more, 50 days or more, 100 days or more. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt, base, acid or amine thereof. In some embodiments, the administering is over a period of months, and the co-therapeutic agent is clobazam. In some embodiments, the co-therapeutic agent is a combination of stiripentol, valproate and clobazam. In some embodiments of the method, the repeating administration continues over a period of 4 weeks or more until a reduction from baseline in a particular seizure type experienced by the patient is observed. In some embodiments, the repeating administration continues until a particular seizure type experienced by the patient is eliminated for a period of 10 days or more. In some embodiments, the repeating administration continues over a period of 4 weeks or more by administering the fenfluramine twice per day in a liquid formulation in an amount of 0.2 mg/kg/day to 5 mg/kg/day until a particular seizure type experienced by the patient is eliminated over a period of 10 days or more. In some aspects, the present disclosure provides a method of treating a patient diagnosed with Dravet syndrome, comprising administering to the patient a therapeutically effective dose of fenfluramine or a pharmaceutically acceptable salt, base, acid or amine thereof in an amount an initial dose ranging from between 0.2 mg/kg/day and 5 mg/kg/day up to a maximum of 60 mg per day for up to about 5 days; administering a co-therapeutic agent; and repeating the administering of the co-therapeutic agent and fenfluramine over a period of weeks until the patient exhibits a reduction from baseline in two types of seizures. In some aspects, provided herein is a use of a formulation for treating a patient experiencing uncontrolled seizures, wherein the formulation comprises a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; a co-therapeutic agent; and wherein the co-therapeutic agent and fenfluramine are in a liquid formulation for use over a period of weeks until the patient exhibits a reduction from baseline of two types of seizures. Pharmaceutical compositions and formulations for use in practicing the subject methods are also provided.

In some embodiments, the method comprises includes a use of a therapeutically effective dose of fenfluramine or a pharmaceutically acceptable salt, base, acid or amine thereof; a concomitant anti-epileptic drug (AED); and wherein the fenfluramine and the AED are in a liquid formulation for use in repeated daily administrations over a period of days while gradually reducing AED administered while maintaining efficacy of treatment.

The invention includes a use of a formulation for treating a patient diagnosed with a refractory epilepsy, wherein the formulation comprises a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; a concomitant anti-epileptic drug (AED); monitoring symptoms of the patient; wherein the fenfluramine and AED are in a liquid formulation for use in repeated daily administrations while gradually reducing AED administered while continuing the monitoring to confirm symptoms are maintained or improved.

In some aspects, provided herein is a method of reducing dosage of a concomitant medication in a human patient diagnosed with Dravet syndrome or other epileptic encephalopathy, by administering to the patient a therapeutically effective dose of fenfluramine or a pharmaceutically acceptable salt, base, acid or amine thereof, and repeating the administering over a period of days while reducing the dose of one or more concomitant anti-seizure or anti-epileptic drugs (AEDs) from baseline and thereby decreasing the amount of medication given to the patient while reducing adverse side effects. In some aspects, provided herein is a method of treating a patient diagnosed with Dravet syndrome by administering to the patient a therapeutically effective dose of fenfluramine or a pharmaceutically acceptable salt, base, acid or amine thereof; administering to the patient over a period of days a concomitant AED; and continuing to administer the fenfluramine and the AED over a period of days while gradually reducing AED administered while maintaining the efficacy of treatment. In some embodiments of the method, the concomitant AED is reduced in increments while monitoring efficacy of the treatment. In some embodiments of the method, the incremental reduction continues over a period of days, or over a period of weeks, or over a period of months. In some embodiments of the method, the reduction continues until the patient no longer receives a dose of the concomitant AED. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, topiramate, valproic acid, valproate, verapamil, and benzodiazepines such as clobazam, clonazepam, diazepam, lorazepam, and midazolam and a pharmaceutically acceptable salt, base, acid or amine thereof. In some embodiments, the administering is over a period of months, and the co-therapeutic agent is clobazam. In some embodiments, the co-therapeutic agent is a combination of stiripentol, valproate and clobazam. In some embodiments, the method further comprises repeating the administration until the clobazam is no longer administered. In some embodiments of the method, the treatment improves two or more symptoms selected from the group consisting of convulsive seizures, ataxias, gait abnormalities, sleep disturbances and cognitive impairment. In some embodiments, the method further comprises repeating the administering of the AED until the amount of AED administered on a daily basis is reduced by 25% or more. In some embodiments, the method further comprises repeating the administering of the AED until the amount of AED administered on a daily basis is reduced by 50% or more. In some embodiments, the method further comprises repeating the administering of the AED until the amount of AED administered on a daily basis is reduced by 75% or more. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some aspects, provided herein is a method of treating a patient diagnosed with refractory epilepsy by administering to the patient over a period of days a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; administering to the patient over a period of days a concomitant anti-epileptic drug (AED); monitoring symptoms of the patient; and continuing to administer the fenfluramine and AED while gradually reducing AED administered while continuing the monitoring to confirm symptoms are maintained or improved. In some embodiments, the refractory epilepsy such as SE, RSE or SRSE which was originally diagnosed as Dravet syndrome, Lennox-Gastaut syndrome, or Doose syndrome and later progressed to SE, RSE or SRSE. In some embodiments, the refractory epilepsy is Dravet syndrome and the fenfluramine and AED are administered twice daily in a liquid formulation. In some embodiments, the fenfluramine is administered in an amount ranging from between 0.2 mg/kg/day and 5 mg/kg/day up to a maximum of 60 mg per day for up to about 5 days. In some aspects, provided herein is a use of formulation for treating a patient diagnosed with a refractory epilepsy, wherein the formulation comprises a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; a concomitant anti-epileptic drug (AED); wherein both the fenfluramine and AED are for use while monitoring symptoms of the patient; and wherein the fenfluramine and AED are used while gradually reducing AED administered while continuing the monitoring to confirm symptoms are maintained or improved. Pharmaceutical compositions and formulations for use in practicing the subject methods are also provided.

In some embodiments, provided herein is a method of treating a selected epileptic patient population having SE, RSE or SRSE and placed in a medically-induced coma via a general anesthetic, wherein the selected patients have been selected based on a determination that the epileptic patients have previously been non-responsive when treated with cannabidiol and/or stiripentol, or the patient's response to cannabidiol and/or stiripentol diminished with increasing time. In some embodiments, the method comprises selecting the patient based on a previously failed treatment with cannabidiol and/or stiripentol, based on lack of efficacy or tolerability. In some embodiments, the method comprises administering to each identified patient in the selected population of patients a therapeutically effective dose of fenfluramine or a pharmaceutically acceptable salt, base, acid or amine thereof, and repeating the administering over a period of a day or days, or over a period of weeks, months or years, until the patient can be weaned from general anesthesia. In some embodiments, the method of treating a patient in a selected patient population having a refractory epilepsy such as SE, RSE or SRSE and having been put into a medically-induced coma via a general anesthetic employs a therapeutically effective dose of fenfluramine or a pharmaceutically acceptable salt, base, acid or amine thereof, in a formulation for administering to a patient previously determined non-responsive when treated with cannabidiol and/or stiripentol, or whose response to cannabidiol and/or stiripentol diminished over time, and the dose is repeated over a period of days until the patient can be weaned from the general anesthetic and released from the medically-induced coma.

The method includes a use of a formulation for treating a patient in a selected patient population having refractory epilepsy such as SE, RSE or SRSE and in a medically-induced coma, the formulation comprising a therapeutically effective dose of fenfluramine or a pharmaceutically acceptable salt, base, acid or amine thereof, wherein the formulation is for use with a patient previously determine non-responsive when treated with cannabidiol and/or stiripentol or the patient's response to cannabidiol and/or stiripentol diminished over time; and wherein the use is repeated over a period of days until the patient can successfully be weaned from the general anesthetic and released from the medically-induced coma.

The method includes a use of a formulation for treating a patient in a selected patient population wherein the patient is diagnosed with Dravet syndrome, Lennox-Gastaut syndrome, or Doose syndrome and later progressed to SE, RSE or SRSE, the use comprising a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; administering a co-therapeutic agent; and wherein the formulation is for use with the patient previously determined non-responsive when treated with cannabidiol and/or stiripentol or the patient's response to cannabidiol and/or stiripentol diminished over time; wherein the use is repeated over a period of weeks until the patient can successfully be weaned from the general anesthetic and released from the medically-induced coma.

In some embodiments of the method, the patient is administered the therapeutically effective dose for a period of weeks/months/years and the reduction from baseline is sustained for a period of weeks/months/years. In some embodiments in which the repeat administration is daily, the administration is once a day, twice a day, three times a day or four times a day. In some embodiments, the dose is provided to the patient at an initial dose ranging from between 0.2 mg/kg/day and 5 mg/kg/day up to a maximum of 60 mg per day for up to about 5 days. In some embodiments, the patient exhibits, post-treatment with fenfluramine and weaning from the general anesthesia, a reduction from baseline in convulsive seizure frequency of 50% or more, 60% or more, 70% or more, 80% or more, 90% or more, 95% or more. In some embodiments, seizures are completely eliminated for 10 days or more, 20 days or more, 30 days or more, 50 days or more, 100 days or more. In some embodiments, the method further comprises repeating the administering until the patient is seizure free for a period of ≥1 day, or for a period of ≥9 days, or for a period of ≥14 days, or for a period of ≥21 days, or for a period of ≥14 weeks, or for a period of ≥6 months, or for a period of ≥1 year. In some embodiments, the method further comprises repeating the administering until the patient is permanently seizure free. In some embodiments, the fenfluramine or pharmaceutically acceptable salt, base, acid or amine thereof is fenfluramine hydrochloride. In some embodiments, the fenfluramine hydrochloride is in a liquid formulation at a concentration of 1.25 mg/ml, 2.5 mg/ml or 5 mg/ml provided at twelve-hour intervals twice a day using an oral syringe graduated for precise measurement of the dose of the liquid formulation, administered alone or with another antiepileptic drug as a co-therapeutic agent. In some embodiments of the method, fenfluramine is the only active ingredient administered to the patient. In some embodiments, the method further comprises administering a co-therapeutic agent. In some embodiments, the co-therapeutic agent is selected from the group consisting of cannabidiol, carbamazepine, ethosuximide, fosphenytoin, lamotrigine, levetiracetam, phenobarbital, stiripentol topiramate, valproic acid, valproate, verapamil, and benzodiazepines such as clobazam, clonazepam, diazepam, lorazepam, and midazolam and a pharmaceutically acceptable salt, base, acid or amine thereof. In some embodiments, the administering is over a period of months, and the co-therapeutic agent is clobazam. In some embodiments, the co-therapeutic agent is a combination of stiripentol, valproate and clobazam. In some embodiments, the treatment improves two or more symptoms selected from the group consisting of convulsive seizures, ataxias, gait abnormalities, sleep disturbances and cognitive impairment.

In some embodiments, the present disclosure provides a method of treating a patient in a selected patient population wherein the patient is experiencing uncontrolled seizures, comprising determining a patient has previously been non-responsive when treated with stiripentol or the patient's response to stiripentol diminished over time; identifying the patient so determined as being non-responsive; administering to the non-responsive patient a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; administering a co-therapeutic agent; and repeating the administering of the co-therapeutic agent and fenfluramine over a period of weeks until the patient exhibits, upon weaning off of the general anesthesia, a reduction from baseline in convulsive seizure frequency of 60% or more. In some aspects, provided herein is a method of adjusting dose of stiripentol in a human patient experiencing uncontrolled seizures, by administering, to a patient receiving stiripentol, a therapeutically effective dose of fenfluramine or a pharmaceutically acceptable salt, base, acid or amine thereof, and thereafter increasing the fenfluramine dosage to 5 mg/kg/day for days 18-24 of fenfluramine therapy; provided that the total dosage of fenfluramine does not exceed 60 mg/day. In some aspects, provided herein is a method of dosing a patient with fenfluramine, wherein the patient is receiving stiripentol therapy and commencing fenfluramine therapy for treating a form of epilepsy, by administering to the patient receiving stiripentol an initial dosage of fenfluramine of, for example 1 mg/kg/day for the first seven days of fenfluramine therapy; increasing the dosage to 5 mg/kg/day for days 8-15 of fenfluramine therapy; provided that the total dosage of fenfluramine does not exceed 60 mg/day. If a treating physician determines that a patient needs a more rapid titration the dose may be increased in increments of not more than 0.2 mg/kg/day every 4 days, up to a dose of 5 mg/kg/day or a maximum dose of 60 mg/day. In some embodiments of these methods, the form of epilepsy is chosen from Dravet syndrome, Lennox-Gastaut syndrome and Doose syndrome. In some embodiments of these methods, the titration provides increased tolerability of the combination of stiripentol and fenfluramine. In some embodiments, the patient is already receiving one or more co-therapeutic agents in addition to stiripentol. In some aspects, provided herein is a use of a formulation for treating a patient in a selected patient population wherein the patient is diagnosed with Dravet syndrome, wherein the formulation comprises a therapeutically effective dose of fenfluramine or a pharmaceutically acceptable salt, base, acid or amine thereof in an amount ranging from between 0.2 mg/kg/day and 5 mg/kg/day up to a maximum of 60 mg per day for up to about 5 days; a co-therapeutic agent; and wherein the formulation is used with a patient previously determined to be non-responsive when treated with stiripentol, or the patient's response to stiripentol diminished over time; wherein the co-therapeutic agent and fenfluramine are for use over a period of weeks until the patient is determined as non-responsive to stiripentol exhibits a reduction from baseline in convulsive seizure frequency of 60% or more. Pharmaceutical compositions and formulations for use in practicing the subject methods are also provided.

In some aspects, provided herein is a method of adjusting dose of cannabidiol or stiripentol in a human patient with SE, RSE or SRSE experiencing uncontrolled seizures and in a medically-induced coma via general anesthesia, by administering a therapeutically effective dose of fenfluramine or a pharmaceutically acceptable salt, base, acid or amine thereof, and increasing the fenfluramine dosage to an initial dose ranging from between 0.2 mg/kg/day of fenfluramine therapy; and thereafter increasing the daily dosage up to 5 mg/kg/day; provided that the total dosage of fenfluramine does not exceed 60 mg/day for up to about 5 days. In some aspects, provided herein is a method of dosing a patient with fenfluramine, wherein the patient is receiving cannabidiol or stiripentol therapy and commencing fenfluramine therapy for treating the SE, RSE or SRSE, by administering to the patient receiving cannabidiol or stiripentol an initial dosage of fenfluramine of 1 mg/kg/day for the first seven days of fenfluramine therapy; and thereafter increasing the daily dosage to 5 mg/kg/day; provided that the total dosage of fenfluramine does not exceed 60 mg/day. If a treating physician determines that a patient needs a more rapid titration the dose may be increased in increments of 0.5 mg/kg/day up to the maximum dose of 60 mg/day. In some embodiments of these methods, the form of epilepsy originally diagnosed is chosen from Dravet syndrome, Lennox-Gastaut syndrome and Doose syndrome. In some embodiments of these methods, the titration provides increased tolerability and/or efficacy of the combination of cannabidiol and fenfluramine. In some embodiments of these methods, the titration provides increased tolerability and/or efficacy of the combination of stiripentol and fenfluramine. In some embodiments, the patient is already receiving one or more co-therapeutic agents in addition to cannabidiol. In some embodiments, the patient is already receiving one or more co-therapeutic agents in addition to stiripentol.

Pharmaceutical compositions and formulations for use in practicing the subject methods are also provided. The formulation may include flavoring and coloring agents or may be completely devoid of any excipient materials beyond those necessary to dissolve the fenfluramine in the liquid which may be water.

In some embodiments, the fenfluramine is adjunctive therapy and is co-administered with a second, or a second and third, or a second, third and fourth, therapeutic agent. Any second, or second and third, or second, third and fourth therapeutic agents may be utilized. In some cases, the additional therapeutic agents are selected from the group consisting of cannabidiol, carbamazepine, ethosuximide, fosphenytoin, lamotrigine, levetiracetam, phenobarbital, topiramate, stiripentol, valproic acid, valproate, verapamil, and benzodiazepines such as clobazam, clonazepam, diazepam, lorazepam, and midazolam and a pharmaceutically acceptable salt, base, acid or amine thereof.

Aspects of the subject methods include identifying a patient previously treated unsuccessfully with stiripentol who will benefit from treatment with fenfluramine according to the methods described herein. Fenfluramine can then be employed to treat the patient either as a subsequent monotherapy or as a co-therapy with stiripentol. In some cases, the patient can be monitored for a reduction in instances of seizures (e.g., mean monthly convulsive seizures) relative to that observed under prior treatment with stiripentol.

Fenfluramine can be employed to treat a patient who has previously been treated with cannabidiol. In some instances, the patient is diagnosed with Dravet syndrome that is refractory to treatment with cannabidiol. By refractory to cannabidiol is meant that the frequency of convulsive seizures (CSF) is not significantly reduced in the patient in response to therapy (e.g., monotherapy) with cannabidiol (CBD). In some cases, a significant reduction in CSF is a 10% or greater reduction in mean monthly convulsive seizures, such as 15% or greater, 20% or greater, 25% or greater, 30% or greater, 35% or greater, 40% or greater, or 45% or greater reduction. In certain instances, the subject method is a method of preventing or treating seizures in a patient diagnosed with Dravet syndrome refractory to treatment with cannabidiol by administering to that patient a therapeutically effective dose of fenfluramine, whereby seizures are prevented or reduced. In various embodiments of this aspect, the instances of seizures (e.g., mean monthly convulsive seizures) are decreased by at least 50%, at least 60%, at least 70%, at least 80% or at least 90%. Aspects of the subject methods include identifying a patient previously treated unsuccessfully with cannabidiol who will benefit from treatment with fenfluramine according to the methods described herein.

Fenfluramine can then be employed to treat the patient either as a subsequent monotherapy or as a co-therapy with a second agent, such as cannabidiol. In some cases, the patient can be monitored for a reduction in instances of seizures (e.g., mean monthly convulsive seizures) relative to that observed under prior treatment with cannabidiol.

Fenfluramine can be administered in the form of the free base, or in the form of a pharmaceutically acceptable salt, base, acid or amine for example selected from the group consisting of hydrochloride, hydrobromide, hydroiodide, maleate, sulphate, tartrate, acetate, citrate, tosylate, succinate, mesylate and besylate. Further illustrative pharmaceutically acceptable salts can be found in Berge et al., J. Pharm. Sci. (1977) 68(1):1-19.

Fenfluramine for use in the methods of the present invention may be produced according to any pharmaceutically acceptable process known to those skilled in the art.

Examples of processes for synthesizing fenfluramine are provided in the following documents: GB1413070, GB1413078 and EP441160. An example of a fenfluramine drug product synthesis is provided in US20180148403.

The dose of fenfluramine to be used in a method of the present invention can be provided in the form of a kit, including instructions for using the dose in one or more of the methods of the present invention. In certain embodiments, the kit can additionally comprise a dosage form comprising one or more co-therapeutic agents. The kit may also contain directions for initiating fenfluramine therapy in a patient, in some instances the direction may take into account co-administration with other interacting antiepileptic drugs and provide alternate dosing instructions when the patient also receives those drugs concomitantly.

A method of the present invention can be practiced on any appropriately diagnosed patient. In a typical embodiment of the present invention, the patient may be an adult, and may be aged about 18 or less, about 16 or less, about 14 or less, about 12 or less, about 10 or less, about 8 or less, about 6 or less or about 4 or less to about 0 months or more, about 1 month or more, about 2 months or more, about 4 months or more, about 6 months or more or about 1 year or more. Thus, the diagnosed patient is typically about one month old or older when treated.

The invention is further illustrated in the following Examples.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Fenfluramine was tested on a 20-year-old female patient identified as “G.R.” with an established diagnosis Dravet Syndrome (DS) (February, 2008) and having undergone confirmatory genetic testing, she was hospitalized in November 2018 and had been in an intensive care unit (ICU) under general anesthesia for management of super-refractory status epilepticus. At the beginning of the testing period, she had been in the ICU for around 32 days, and multiple attempts to wean her from anesthetic agents (pentobarbital and ketamine) were unsuccessful, with repetitive and then continuous tonic seizures recurring within hours. Table 1, below, provides a complete list of all anti-epileptic drugs (AEDs) assayed in managing the patient's seizures. Some had been determined to lack efficacy, while those marked as current were still being administered.

TABLE 1
Prior failed AEDs (complete list)

a. Phenobarbital (1998, 2002, 2005, current)
b. Tegretol (1998)
c. Lamictal (1999)
d. Klonopin (1999, current)
e. Keppra (1999, 2015-current)
f. Dilantin (2002)
g. Depakote (2002, 2008-current)
h. Felbatol (2002)
i. Zonegran (2003)
j. Lyrica (2005)
k. Frisium/Onfi (2007, 2010-current)
l. Diacomit (2009)
m. Elavil (2010)
n. Topamax (2010)
o. Epidiolex (2018-current)
p. Ativan (1999)
q. Diastat (1999-current) (home administrable valium rescue drug)
r. Ketogenic Diet (2000, 2013, 2015)
s. VNS (2003-current)

The treating physician had made rapid trials of some of these AEDs, watching for a clinical effect upon rapid weaning—from anesthetic agents.

After these other AEDs failed, the attending physician tested fenfluramine, beginning on Dec. 19, 2018. A baseline echocardiogram was taken with the patient still in ICU setting. The patient's medications (beyond anesthetic agents) at that time included valproic acid, levetiracetam, clobazam, phenobarbital, ketosis, and VNS therapy, and felbamate was most recently added after failure of the last attempt to withdraw anesthesia.

Fenfluramine was administered as an oral solution via a gastric feeding tube at an initial dose of 0.5 mg/kg/day for about 5 days. An echocardiogram was performed to screen for cardiac valve abnormalities (CVD) and/or pulmonary arterial hypertension (PAH) and to establish a baseline for subsequent echocardiograms which will be used to monitor the patient's cardiovascular status over the treatment period, and repeated approximately every 3 months over the next year of continuing fenfluramine administration. Markers of fenfluramine efficacy were improved seizure control following wean-off of anesthetic agents (pentobarbital and ketamine). Previous attempts to wean the patient from anesthetics lead to break-through seizures every 3-7 minutes towards end of weaning process which resulted in re-establishment of the induced coma. After 4 days of fenfluramine administration Patient G.R. was able to be weaned from general anesthesia without return to seizures and the immediate crisis resolved, however, seizures began to recur after 3 more days without anesthesia and the fenfluramine dosage was adjusted up to 0.8 mg/kg/day and bolus doses of valproate or phenobarbital were administered as needed. The treating physician assessed the patient as having emerged from the SRSE crisis about 2 weeks after initiation of fenfluramine therapy. The patient remains on fenfluramine, along with levetiracetam, clobazam and valproate, and was discharged from the ICU about 1 month (approximately 32 days) after initiation of fenfluramine therapy. Serum samples were obtained and analyzed, and results presented in the table below.

| | | Time- | | Conc. | | |
|---|---|---|---|---|---|---|
| SUB | Visit | point | Analyte | (ng/mL) | Batch | SPECCOND |
| 001 | 1 | 99 | Fenfluramine | 17.7 | 2 | SAE 8 Jan. 2019 |
| 001 | 1 | 99 | Norfenflur- | 23.8 | 2 | SAE 8 Jan. 2019 |
| | | | amine | | | |
| 001 | 2 | 99 | Fenfluramine | 17.8 | 2 | SAE 9 Jan. 2019 |
| 001 | 2 | 99 | Norfenflur- | 23.1 | 2 | SAE 9 Jan. 2019 |
| | | | amine | | | |
| 001 | 3 | 99 | Fenfluramine | 22.2 | 2 | SAE 10 Jan. 2019 |
| 001 | 3 | 99 | Norfenflur- | 27.6 | 2 | SAE 10 Jan. 2019 |
| | | | amine | | | |

These results confirm that fenfluramine provides improved seizure control in patients with SRSE.

The preceding disclosure merely illustrates principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of improving seizure control in a patient experiencing uncontrolled seizures persisting 10 mins or more, comprising:
administering fenfluramine or a pharmaceutically acceptable salt, base, acid or amine thereof, at a dose of 0.2 to 1 mg/kg/day and/or for a period of from 12 hours to about 7 days to a patient in a medically-induced coma via a general anesthetic; and
after 12 hours to about 7 days, weaning the patient from the general anesthetic and assessing whether the seizure control has improved.

2. The method of claim 1, wherein the assessing of seizure control is measured by occurrence of break-through seizures appearing on one or more of electroencephalographic monitoring, video-electroencephalographic telemetry and continuous video observation by trained personnel, or any combination thereof;
wherein the weaning is started at about 1 to about 4 days after starting administration of fenfluramine;
the assessment indicates that seizure control has not improved adequately, and
the method further comprises reestablishing the medically-induced coma with a general anesthetic, and
increasing the dose of fenfluramine;
wherein the general anesthetic is chosen from propofol, a barbiturate, midazolam and ketamine, or any combination thereof.

3.-6. (canceled)

7. The method of claim 1, wherein seizure control has improved sufficiently to continue weaning the patient from general anesthesia while continuing to administer fenfluramine;
wherein the continuing administration of fenfluramine is titrated to between 0.2 mg/kg/day and 0.8 mg/kg/day.

8.-11. (canceled)

12. The method of claim 1, wherein the patient has been diagnosed with a disease or condition selected from an epilepsy or epileptic encephalopathy (e.g., Dravet syndrome, Doose syndrome, infantile spasms, Lennox-Gastaut syndrome, Rett syndrome); attentional disorders (e.g., attention deficit disorder (ADD) or attention deficit/hyperactivity disorder (ADHD)); developmental disorders, such as autism spectrum disorders (ASDs), including autism, Asperger syndrome, pervasive developmental disorder (PDD) and pervasive developmental disorder not otherwise specified (PDD-NOS); oppositional defiant disorder (ODD); learning disabilities (e.g. dyslexia, dyscalculia); Tourette syndrome; traumatic brain injury; lead exposure; anxiety and/or depression; and low birth weight; or any combination thereof;
wherein the fenfluramine is formulated with a pharmaceutically acceptable carrier, and an effective dose is selected from less than about 10.0 mg/kg/day, less than 1.0 mg/kg/day, about 0.8 mg/kg/day, and about 0.5 mg/kg/day.

13. The method of claim 1, wherein the patient has been diagnosed with Dravet syndrome.

14. The method of claim 1, wherein the patient has been diagnosed with Lennox-Gastaut syndrome.

15. (canceled)

16. The method of claim 1, wherein fenfluramine is used as an adjunctive therapy in a patient with Dravet syndrome or Lennox-Gastault syndrome (LGS).

17. The method of claim 1, wherein at least one co-therapeutic agent is administered, and wherein said agent is selected from the group consisting of Brivaracetam, bromides (e.g., Potassium Bromide, Sodium Bromide), Cannabidiol, Carbamazepine, Clonidine, Ergenyl Chrono, Ethosuximide, Felbamate, Fosphenytoin, Ketamine, Lacosamide, Lamotrigine, Levetiracetam, Levocarnitine, Mesuximide, Nitrazepam, Oxcarbazepine, Perampanel, Phenobarbital, Pregabalin, Progabide, Pyridoxine, Rufinamide, Sultiame, Tizanidine, Topiramate, Stiripentol, Valproate semisodium, Valproate sodium, Valproic acid, Verapamil, Zonisamide, and benzodiazepines such as Clobazam, Clonazepam, Diazepam, Ethyl Loflazepate, Lorazepam and Midazolam, and a pharmaceutically acceptable salt, base, acid or amine thereof.

18-21. (canceled)

22. The method of claim 1 wherein the fenfluramine is in an oral solution administered via a gastric feeding tube.

23. The method of claim 22, wherein the improved seizure control is observed as a decrease in frequency, length or severity, and/or an increase in the length of time between seizures, as compared to the frequency, length, severity, or length of time between seizures in a patient not treated with fenfluramine and weaned from general anesthesia.

24.-42. (canceled)
